(19) United States
(12) Patent Application Publication
(10) Pub. No.: US 2011/0166438 A1
(43) Pub. Date: Jul. 7, 2011

(54) RF FIELD SHAPING AND ATTENUATION FOR EMAI INDUCTION ELEMENTS

(76) Inventors: Jane F. Emerson, Irvine, CA (US); David B. Chang, Tustin, CA (US); Stephen A. Cerwin, Mico, TX (US); James E. Drummond, Lincoln City, OR (US); Joy Drummond, Lincoln city, OR (US)

(21) Appl. No.: 12/896,733

(22) Filed: Oct. 1, 2010

Related U.S. Application Data
(60) Provisional application No. 61/287,305, filed on Dec. 17, 2009.

(51) Int. Cl. A61B 8/13; A61B 8/00; A61B 5/055

(52) U.S. Cl. 600/411; 600/407; 600/443

(57) ABSTRACT

An Electro-Magnetic Imaging (EMAI) System is presented. EMAI systems can include induction elements (e.g., an induction coil) configured to induce a target tissue to generate internally sourced ultrasounds. The induction elements can be shielded by one or more shielding elements to shape, or otherwise alter, an imaging field while attenuating radiated fields in a far zone. EMAI systems can further include a shield tuner to adjust shield parameters to achieved desired imaging or radiated field properties. A shielding element can be placed approximately one induction coil radius away from the coil to achieve suitably strong imaging field magnitudes while also achieving suitably weak radiated field magnitudes in a far zone. In some embodiments, acoustic sensors lack substantial shielding from the fields generated by the induction elements.

This application claims the benefit of priority to U.S. provisional application having Ser. No. 61/287,305 filed on Dec. 17, 2009. This and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

### FIELD OF THE INVENTION

The field of the invention is electromagnetic field adjusting technologies.

### BACKGROUND

Magnetic Resonance Imaging (MRI) apparatus employ one or more induction coils to emit electromagnetic fields into tissues of a patient. Reflections of the fields from the tissue can be used to generate image data relating to a target tissue.

A lesser known technique for imaging is known as “Electro-Magnetic Acoustic Imaging” (EMAI) where electromagnetic fields bathe a target tissue. The fields can induce the target tissue to generate an ultrasound or acoustic signal due to conductivity gradients present in the tissue observation area. The acoustic signals originating from the tissue can also be used to generate images of the target tissue. Such approaches are described in the Applicant's previous patent filings including U.S. Pat. No. 6,535,625 to Chang et al. titled “Magneto-Acoustic Imaging” filed Sep. 24, 1999, and U.S. Pat. No. 6,974,415 to Cerwin et al. titled “Electromagnetic-Acoustic Imaging” filed on May 23, 2003.

Interestingly, MRI apparatus use Radio Frequency (RF) shielding to reduce acoustic noise in the MRI apparatus, or other objects. For example, RF shielding is used to reduce induced eddy currents caused by the MRI coils, where the eddy currents are induced in other shielding, equipment, or other objects. The eddy currents cause the equipment to vibrate generating undesired acoustic noise. U.S. Pat. No. 7,372,271 to Roozen et al. titled “Main Magnet Perforated Eddy Current Shield for a Magnetic Resonance Imaging Device”, filed Mar. 8, 2005, describes shields to reduce eddy currents to prevent acoustic noise. Another example includes U.S. Pat. No. 7,375,526 to Edelstein et al. titled “Active-Passive Electromagnetic Shielding to Reduce MRI Acoustic Noise”, filed on Oct. 20, 2006. Yet another example includes U.S. Pat. No. 4,737,716 to Roemer et al. titled “Self-Shielded Gradient Coils for Nuclear Magnetic Resonance Imaging”, filed Feb. 6, 1986. Similarly, international patent application publication WO 91/12209 to Rzedzian et al. titled “Shielded Gradient Coils for Nuclear Magnetic Resonance Imaging”, filed Jun. 6, 1991, also described shielded coils.

Even in the Applicant's own work described in U.S. patent application 2007/0038060 to Cerwin at al. titled “Identifying and Treating Bodily Tissues using Electromagnetically Induced, Time-Reversed, Acoustic Signals”, filed Jun. 9, 2006, it has been discussed that shielding is desirable to protect an ultrasonic sensor array. In a somewhat similar vein, U.S. patent application publication 2007/0167705 to Chaing et al. titled “Integrated Ultrasound Imaging System”, filed Aug. 2, 2006, discusses an ultrasound and MRI system where the ultrasound transducer or other components are shielded from electromagnetic interference.

A more ideal approach in an imaging solution with shielding would offer field shaping toward a target tissue in addition to providing shielding for far field attenuation. Typically, imaging techniques utilize multiple induction coils to achieve a desired field shape at a target tissue site. For example, U.S. patent application publication 2005/0205566 to Kassayan titled “System and Method of Interferentially Varying Electromagnetic Near Field Patterns”, filed May 27, 2004, discusses using near field shaping antennas to reduce electromagnetic field near the housing of a device. Unfortunately, the Kassayan approach can be invasive and not a practical solution for EMAI applications.

Although a great deal of effort has been directed toward providing shielding against undesirable induced acoustic noise, it has yet to be appreciated that induced acoustic noise can be desirable within a target tissue, especially in EMAI applications. RF shielding can be used to attenuate undesirable far fields while also enhancing induced acoustic signals in a target tissue, possible through field shaping achieved by adjusting shielding parameters.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

Thus, there is still a need for shielding for induction elements.

### SUMMARY OF THE INVENTION

The inventive subject matter provides apparatus, systems and methods in which an EMAI system includes an induction element, an element shield, and an acoustic sensor. The element shield can be configured to attenuate undesirable electromagnetic (EM) fields in a direction (e.g., far fields) away from a target tissue, while the element can actively or passively shape the EM fields directed to the target tissue. The shaped EM fields can be directed toward the target tissue, where the fields induce an acoustic signal in the tissue as a result of conductivity gradients in the tissue. The acoustic sensor of the EMAI system can capture the internally sourced acoustic signals originating from the target tissue. The acoustic signals can then be converted to image data suitable for display.

In some embodiments, the inductive elements can comprise one or more EM coils. More preferred coils include Helmholtz coils or pancake coils. Additionally, the shields and coils or other induction elements and can be spatially adjusted relative to each other to tune the system to adjust one or more properties of the shaped EM fields. It is also contemplated that tuning the system can occur automatically under direction of one or more computing devices.

As used herein, and unless the context dictates otherwise, the term “coupled to” is intended to include both direct coupling (i.e., in which two elements that are coupled to each other are in contact with each other) and indirect coupling (i.e., in which at least one additional element is located between the two elements). Therefore, the terms “coupled to” and “coupled with” are used synonymously.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic overview of an electromagnetic acoustic imaging system having a shield configured to shape emitted electromagnetic fields.

FIG. 2 is schematic overview of an electromagnetic acoustic imaging system having a tuner.

FIG. 3 is a graph of an axial magnetic field based on a Helmholtz coil embodiment.

### DETAILED DESCRIPTION

It should be noted that while the following description is drawn to a electromagnetic acoustic imaging (EMAI) systems, one should appreciate the various device elements, components, modules, or other members of the system can comprise one or computing devices interfaces, databases, engines, controller, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclose apparatus.

One should appreciate that the disclosed techniques provide many advantageous technical effects including enhancing electromagnetic image fields (e.g., near field) for an EMAI system while also attenuating electromagnetic radiated fields (e.g., far fields).

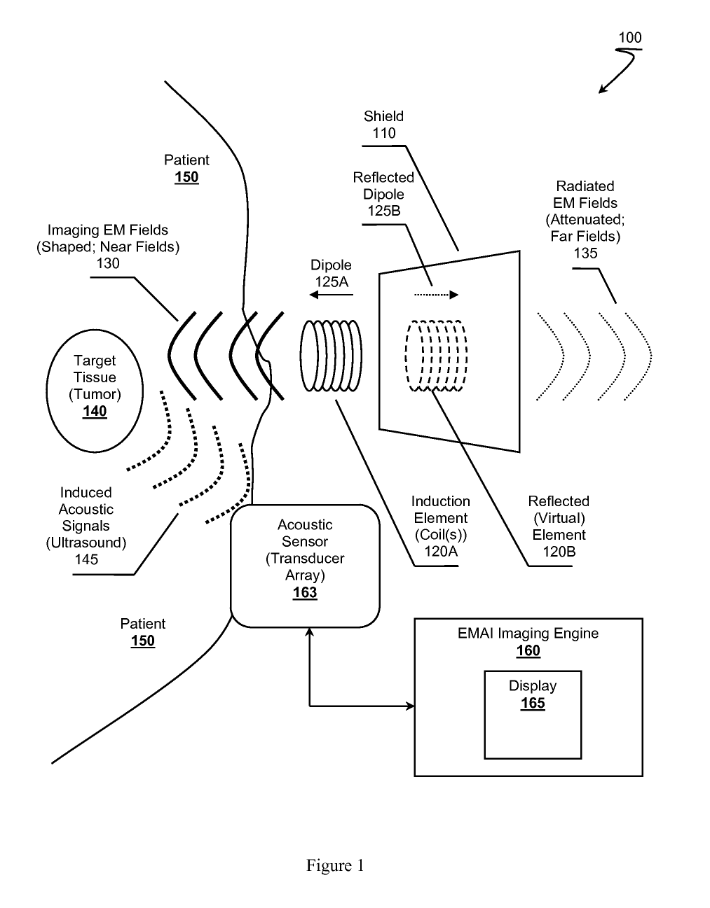

FIG. 1 provides an overview of EMAI system 100 that includes element shield 110. System 100 preferably includes induction element 120A capable of generating radio frequency (RF) EM fields directed toward patient 150 or more preferably toward target tissue 140. The emitted EM fields directed to patient 150 are considered near fields or imaging EM fields 130. Imaging EM fields 130 induce target tissue 140 to generate one or more acoustic signals 145 in response to imaging EM fields 130 impinging on conductivity gradients within target tissue 140. Acoustic signals 145, preferably comprising ultrasound signals, travel through patient 150 and can be collected by one or more of acoustic sensor 163. In more preferred embodiments, acoustic sensor 163 comprises a transducer array capable of generating as well as receiving ultrasound signals. Acoustic data representing acoustic signals 145 can be sent to EMAI imaging engine 160, which in turn converts the acoustic data into imaging data suitable for display on display 165. Acquisition and display of images resulting from acoustic signals 145 is discussed in the Applicant's previous filings including U.S. Pat. No. 6,535,625; U.S. Pat. No. 6,974,415; and U.S. patent application publication 2007/0038060.

Induction elements 120A can take on many different forms depending on the desired properties of system 100. For example, induction elements 120A can comprise induction coils including flat (e.g., pancake) coils, crescent coils, Helmholtz coils, or other types of coils configured to emit imaging EM fields 130. Induction elements 120A can also include an antenna array, possibly comprising a ferrite antenna array. Although induction elements 120A are represented by a single coil, one should appreciate that multiple coils or elements can be used generated desired EM fields 130. In some embodiments, induction elements 120A provide RF fields at or below 14 MHz. One preferred operating range for elements can include 5 MHz to 10 MHz range. Other embodiments can be constructed where induction elements 120A could operate in accordance with Industrial, Scientific, or Medical (ISM) bands as suggested by ITU-R. For example, induction elements 120A could operate near 6.78 MHz or 13.56 MHz. It is further contemplated that an EMAI system could be adapted to operate at frequencies greater than 14 MHz, possibly as part of an MRI apparatus, or even operate in microwave regions.

The electromagnetic fields emitted by induction element 120A can comprise a single frequency or multiple frequencies as desired. In some embodiments, it is advantageous to emit two or more, non-harmonic frequencies so that resulting acoustic signals 145 can be easily separated and identified. For example, two input frequencies could be a 2 MHz and 7 MHz. The acoustic signals could be detected by receiving the sum or difference of the frequencies (i.e., 9 MHz and 5 MHz), possibly in addition to the typically induced ultrasound signals at twice the input frequencies (i.e., 4 MHz and 14 MHz).

EMAI system 100 can utilize one or more MRI coils configured to provide imaging EM fields 130. For example, an MRI apparatus can be adapted to provide support for EMAI applications. Possible candidate MRI coils that could be adapted for use include those described in U.S. Pat. No. 5,530,355 to Doty titled “Solenoidal, Octopolar, Transverse Radiant Coils”, filed May 13, 1993; U.S. Pat. No. 5,554,929 to Doty et al. titled “Crescent Gradient Coils”, filed Mar. 12, 1993; U.S. Pat. No. 5,561,371 to Schenck titled “Transverse Gradient Coil”, filed Sep. 27, 1995; and U.S. Pat. No. 5,886,548 to Doty et al. titled “Crescent Gradient Coils”, filed Feb. 29, 1996.

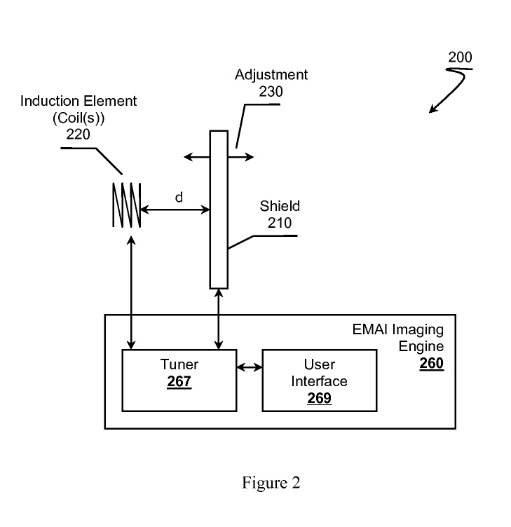

In more preferred embodiments, EMAI system 100 also includes element shield 110. Preferred element shields attenuate undesired EM far fields away from target tissue 140 as represented by radiated EM fields 135. In some embodiments, element shield 110 attenuate the EM far field sufficiently to comply with one or more standards or regulations for radiating medical equipment. In addition to attenuate the far fields element shield 110 can also be used to shape the emitted near EM fields (e.g., imaging EM field 130) originating from induction element 120, possibly enhancing the near fields impinging on target tissue 140. In more preferred embodiments, imaging EM field 130 (e.g., fields focused on a target tissue) can be shaped via appropriate configuration of induction element 120A relative to one or more of shield element 110. For example, an induction coil's properties can be tuned or a density of an induction antenna array can be adjusted, possibly automatically by a tuner (see discussion with respect to FIG. 2).

As briefly mentioned above, inductive element 120A that comprise an antenna array that can be used to shape emitted EM fields, both near (e.g., imaging EM fields 130) or far fields (e.g., radiated EM field 135), through suitable adjustment of each array elements phase or amplitude. Such shaped fields can be either attenuated or enhanced through constructive or destructive interference.

Element shield 110 can comprise one or more conductive or high dielectric plates, possibly with holes, to attenuate or shape the emitted EM fields originating from induction element 120A. Conductive plates could provide for a reflected or virtual image of inductive elements 120A within the plate as represented by reflected element 120B. Virtual element 120B can be considered to at least partially repel, or partially reflect, the emitted EM field back toward patient 150, thus shaping the EM fields into imaging EM field 130. It is noted that a reflected field can have opposite phase thus weakening the original field while shaping it. Euphemistically, the reflection property of shield 110 is represented by reflected dipole 125B, which is a reflection of dipole 125A loosely representing induction element 120A. Such effects are dependent on wavelength, geometry of the shield, electrical properties (e.g., conductivity, permeability, permittivity, etc.), or other attributes of shield 110.

In embodiments where system 100 utilizes coils for induction element 120A, the coil shields preferably have a linear dimension to cover the emitted field regions beyond shield 110 away from induction element 120A. Preferred shields have a linear dimension that is at least greater than 0.3 radii of the coil. The linear dimension can also be greater than 1, 2, 3, or possibly greater multiples of coil radius to reduce field leakage under the far field conditions.

As mentioned above shield can be required to comply with one or more standards, regulations, rules, laws, or other types of policies governing use of radiating medical equipment. The policies can also vary from jurisdiction-to-jurisdiction, country-to-country, state-to-state, institution-to-institution, or even from room-to-room in an institution. Therefore, the shielding can be adjusted as necessary to comply with the various policies, possibly to avoid interference with RF communications. In some circumstances such policies can stated in terms of the allowable magnitude of the electric fields at a location in the “far zone” of an inductive element system, where the distance from the inductive element system is large compared with both the dimensions of the inductive element system and the wavelength of the radiation at the frequency at which the system is operating. This is in contrast to the desired RF fields generated by inductive elements 120A in an imaging region of the EMAI system 100 (e.g., near field): there the imaging dimensions are small compared to the wavelength. Shields 110 can be constructed so as to maintain an imaging RF field 130 of adequate magnitude in the near zone toward tissue 140, while at the same time decreasing the radiated EM field 135 in the far zone in order to satisfy any governing EM radiation policies. This is done by adjusting the distance between shields 110 and inductive elements 120A: the larger this distance is the larger will be the imaging EM field 130 in the near zone. At the same time, however, the larger this distance is, the larger will be the radiated EM field 135 in the far zone. Hence, a compromise must be reached: the distance should be chosen sufficiently large so that the near imaging EM field 130 has an acceptably large magnitude but not so large that the far radiated EM field 135 has a too large a magnitude that might violate a governing policy.

It is easy to estimate the magnitude of radiated EM field 135 since both the dimensions of the inductive element 120A and the wavelength are small compared to the distance between inductive element 120A and the far field observation point of interest for the regulations. Thus, for the far field, the inductive element system without a shield can be represented by a magnetic dipole of magnitude M. Suppose now shield 110 is located at a distance d from the inductive elements 120A. The magnetic dipole 125A is now joined by a “virtual” magnetic dipole 125B of opposite sense, located at a distance d on the other side of shield 110. Thus, the radiated field 135 in the far zone is now due to a magnetic quadrupole of approximate magnitude Q=Md. The electric field E at a distance R from the system in the far zone has therefore been reduced from one having an approximate magnitude E=(ω²/c²) M/R to one having an approximate magnitude E=(ω³/c³)Q/R=(ω²/c²)(ω/c) M/R, where ω is the angular frequency of the RF energy and c is the speed of light. In other words, the electric field magnitude has been reduced by a factor on the order of d/λ, where d is the distance of shield 110 from induction element 120A and λ is the wavelength of the RF energy. The reduction is larger when d is smaller.

The magnetic field magnitude of the imaging EM field 130 in the imaging region due to the magnetic moment M is approximately B=M/r³, where r is roughly the linear dimension of the induction element itself. This assumes that the size of the region being imaged is comparable to the size of induction element 120A. When shield 110 is located at a distance d from the magnetic moment, imagine EM field 130 in the near zone is reduced to B=M/r³−M/(r+2d)³, since the effect of shield 110 is to create virtual magnetic moment/dipole 125B of the opposite sense at a distance d behind shield 110. Thus, the larger d is, the less the magnitude of the imaging EM field 130 at the near field is reduced. For example, if d<<r, this expression can be approximated by B=6Md/r⁴.

To obtain a realistic estimate of a reasonable value for d, consider the example of a Helmholtz coil system of two N-turn coils of radius a separated by a gap also equal to a. The axial magnetic field at a point (r, φ, z) due to these two coils with no shields is given by:

B_{zHelmholtz}(r, φ, z)=B_{z}(r, φ, z)+B_{z}(r, φ, a−z) [1]

where

B_{z}(r, φ, z)=[(μNI)/(2π)][1/((a+r)²+z²)^{1/2}][K(m)+{(a²−r²−z²)/((a−r)²⁺z²)}E(m)] [2]

m=4ar[(a+r)²+z²]⁻¹ [3]

and E(m) and K(m) are the complete elliptic integrals of the first and second kind Here I is the current flowing in the coil, ω is its angular frequency, and μ is the permeability of free space.

When shields are added at a distance d behind each coil, the magnetic field is reduced from that in eq. [1] to

B_{zHelmholtzShielded}(r, φ, z)=B_{z}(r, φ, z)+B_{z}(r, φ, a−z)−B_{z}(r, φ, z+2d)−B_{z}(r, φ, a+2d−z) [4]

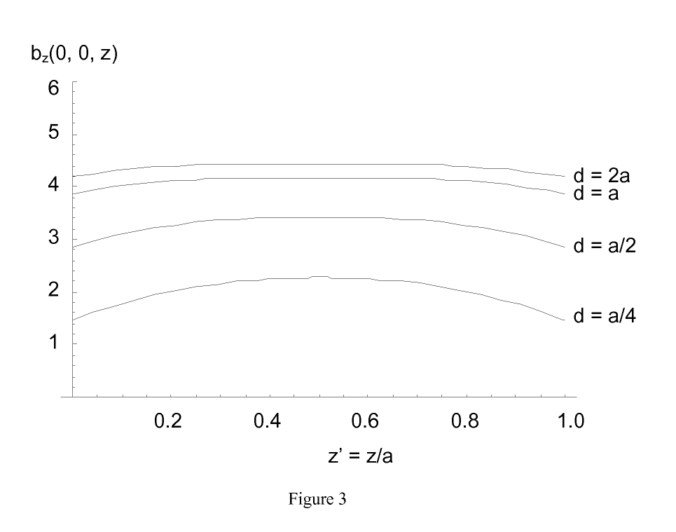

FIG. 3 displays B_{zHelmholtzShielded}(r, φ, z) along the axis of the Helmholtz system (i.e. B_{zHelmholtzShielded}(0, 0, z)) vs. the dimensionless coordinate z′ for different values of d, the distance of the shields behind each of the two coils. From top to bottom, the curves show the field along the system axis for d=2a, a, a/2, and a/4, respectively. This illustrates that when d is small, the imaging field 130 is diminished to a large degree, but that at larger d, the imaging field 130 maintains an acceptable magnitude.

To obtain universal curves, FIG. 3 plots the dimensionless field:

b_{z}(0, 0, z)=[(2πa)/(μNI)]B_{zHelmholtzShielded}(0, 0, z) [5]

against the dimensionless coordinate

z′=z/a [6]

It is apparent from the plots of FIG. 3 that when shield 110 is located much less than a coil radius from a coil, the axial magnetic field is diminished considerably, but when the shield is equal to or larger than a coil radius from the coil, the axial magnetic field is close to its value without the shield. When the distance is increased greater than a coil radius, the gain in the magnitude of the near field is not very great. Hence, a reasonable distance to locate the shield behind the induction coil is on the order of a coil radius.

Although only the axial magnetic field is shown in FIG. 3, the same sort of behavior occurs with the radial magnetic field and azimuthal induction electric fields as well.

The radiated field 135 is decreased considerably with this choice of d. For example, suppose the dimension of the coil is 3 cm, and the RF frequency is 5 MHz. The wavelength is 60 meters, so the radiation field is reduced by a factor of the order of 3/6000=0.0005, which represents approximately a 99.95% reduction. For reasonable coil currents, this reduction attenuates radiated field 135 to a desirable low level, preferably by at least 99%. Naturally, the above example can be adjusted as desired to ensure radiate field from system 100 complies with any governing policies relating to radiating medical equipment.

Thus, a distance d equal to the coil radius will give both an acceptably large imaging field 130 and an acceptably small radiated field 135. Thus shield 110 can be positioned approximately a distance approximately equal to a linear coil dimension, preferably a coil radius, on an opposing side of inductive element 120A relative to target.

Regions of interest for imaging tissue 140 accessible by ultrasound are typically less than 10 cm below the surface of patient 150. As imaging EM field 130 is typically adequately uniform to a depth on the order of the coil radius, then inductive element 120A and reflective elements 120B can preferably have dimensions up to 10 cm. The spacing of the reflective elements 120B can be adjusted according to the above discussion by adjusting properties of shield 110.

FIG. 2 illustrates another possible embodiment represented by EMAI system 200. EMAI system 200 can include shield tuner 267 capable of adjusting one or more parameters of an element shield 210 including spatial separation or orientation of shield 210 relative to induction element 220. As shown, tuner 267 can automatically adjust a separation distance d between element 220 and shield 210. Adjustment of the properties of shield 210 can provide refinement of the imaging EM field shape, magnitude, direction, or other field parameter. The distance d is used euphuistically to represent spatial dimensions or orientations.

It should be appreciated that system 200 can include more than one of element 220 or shield 210. Tuner 267 can be configured to control the various parameters associated within such a system by adjusting each element 220 or shield 210 independently, collectively, or automatically, possibly under programmatic control of EMAI imaging engine 260. In addition, EMAI imaging engine 260 can include user interface 269, through which a user can provide input into tuner 267 to provide for adjustments 230 effecting shield 210 as a single unit or collectively as multiple shield elements. For example, adjustments 230 can include shield separation d as discussed previously, or can also include shape of shield 210 (e.g., concave, convex, parabolic, etc.), angular orientation relative to induction elements 220 or target tissue, movement (e.g., period changes in time of parameters), or other types of adjustments.

Adjustments 230 can also include changes to other parameters of shield 210 beyond physical changes. Other contemplated shield parameters can include electrical parameters (e.g., permittivity, permeability, conductivity, induction, etc.), active field parameters (e.g., phase, polarization, amplitude, etc.), or the types of non-physical parameters. One should note that such changes can be achieved through the use of active shields 210 where the shield itself can be another coil or active element having an inverted sense relative inductive element 220. Even in the passive shield case, EMAI system could swap on type of shield for another type of shield having different parameters.

As intimated above, element shield 210 can be configured to operate as a passive shield or an active shield. A passive shield, possibly a conductor plate, merely shapes the emitted EM fields by the nature of the materials of the shield. An active shield represents a powered source of additional EM fields that enhance, augment, decrease, refine, or otherwise modify the EM fields impinging on a target tissue.

From the earlier discussion associated with the passive shield, it is apparent that the virtual image of the induction coils in the passive shield can always be replaced by active induction coils. To obtain the desired large near imaging fields while also decreasing the radiated EM fields to acceptably low magnitudes, the parameters of the active shield could simply mimic those of the virtual images in the passive shield.

Use of an active shield rather than a passive shield will involve additional energy resources. On the other hand, use of an active shield does permit more flexibility in shaping the near imaging EM fields. This can be done without negatively impacting the desired decrease in the radiation field as long as the active shield elements are arranged so that its associated magnetic dipole elements combine with those of the original system in such a way that the resulting radiation is of a quadrupole nature rather than of a dipole nature.

An astute reader will appreciate FIG. 1 and FIG. 2 illustrate EMAI systems where an acoustic sensor lacks significant shielding, counter to traditional approaches. Such an arrangement is by design to allow the acoustic sensor (e.g., an ultrasound transducer) to generate induced ultrasound signals resulting from the imaging EM field impinging on the acoustic sensor. Such induced ultrasound signals can also be used to target a tissue area. Transducers based on piezoelectric elements can generate such induced ultrasound signals.

Although FIG. 1 and FIG. 2 illustrate a single element with a single element shield, it should be appreciated that an EMAI system can comprise multiple induction elements 220 or modular shields 210 in various configurations as dictated by design requirements. It should also be appreciated that a system can comprise a heterogeneous mix of induction elements (e.g., coils and arrays) to provide desirable field magnitudes or focusing. In fact, the inventive subject matter is considered to include tuning a heterogeneous mix of shielding elements collectively to shape an imaging EM field, which induces conductivity-based acoustic signals within a target tissue. It is also considered advantageous to employ the disclosed techniques for inducing thermo-acoustic signals in a target tissue, possibly based on microwave imagining fields.

The disclosed techniques can be advantageously applied to diagnostic as well as therapeutic EMAI applications. For example, shielding can be used to shape or focus imaging EM fields to properly target a tissue whose induced ultrasound signals can be time-reversed mirrored (TRM) back to the target tissue. The TRM signal can have its gain increased to ablate the target tissue as discussed in the Applicant's co-pending U.S. patent application having Ser. No. 12/786,232 titled “Time-Reversed Mirroring Electro-Magnetic Acoustic Treatment System”, filed on May 24, 2010.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. An electromagnetic acoustic imaging system, comprising:
an induction element configured to emit electromagnetic fields in a first direction toward a target;
an element shield configured to attenuate the electromagnetic fields in at least a second direction away from the target and configured to shape the emitted fields in the first direction; and
an acoustic sensor configured to receive acoustic signals from the target that are induced by the shaped electromagnetic fields.

2. The system of claim 1, wherein the induction element comprises an antenna array.

3. The system of claim 1, further comprising additional element shields that attenuate fields from multiple induction elements.

4. The system of claim 1, wherein the induction element is selected from the group consisting of a Helmholtz coil and a pancake coil.

5. The system of claim 1, wherein the element shield is spatially adjustable relative to the induction element.

6. The system of claim 5, further comprising a shield tuner configured to automatically adjust properties of the element shield.

7. The system of claim 1, wherein the element shield comprises at least one of a conductive plate, and a high dielectric plate.

8. The system of claim 1, wherein the element shield includes holes.

9. The system of claim 1, wherein the induction element comprises a coil.

10. The system of claim 9, wherein the coil is an element of an MRI apparatus.

11. The system of claim 9, wherein the element shield is positioned approximately a coil radius away from the coil on an opposing side of the coil relative to the target.

12. The system of claim 1, wherein the induction element operates at less then 14 MHz.

13. The system of claim 12, wherein the induction element operates at less than 7 MHz.

14. The system of claim 1, wherein the element shield actively shapes the emitted fields.

15. The system of claim 1, wherein the element shield passively shapes the emitted fields.

16. The system of claim 1, wherein the second direction comprises a far field region at a distance greater than roughly a linear dimension of the induction element.

17. The system of claim 16, wherein the electromagnetic field's magnitude is attenuated by at least 99% in the far region.

18. The system of claim 1, wherein the acoustic sensor comprises an ultrasound transducer array.

19. The system of claim 18, wherein the acoustic sensors lacks substantial shielding from the shaped fields.
